(19) United States
(12) Patent Application Publication
(10) Pub. No.: US 2015/0164344 A1
(43) Pub. Date: Jun. 18, 2015

(54) OMNISIGN MEDICAL DEVICE SYSTEMS

(71) Applicant: Jassin M. Jouria, Hollywood, FL (US)

(72) Inventor: Jassin M. Jouria, Hollywood, FL (US)

(21) Appl. No.: 14/588,709

(22) Filed: Jan. 2, 2015

Related U.S. Application Data
(63) Continuation of application No. 13/815,511, filed on Mar. 8, 2013, now abandoned.

(51) Int. Cl. A61B 5/0205; A61B 5/00

(52) U.S. Cl. CPC A61B 5/02055; A61B 5/742; A61B 5/6831; A61B 5/0022; A61B 5/02438

(57) ABSTRACT

A portable medical device is able to measure all four major vital signs and able to wirelessly communicate medical data in real-time to a user. The system includes an arm cuff air bladder of a vital sign detection assembly which may be worn about a forearm of the user. The arm cuff air bladder comprises several sensors in touch-contact with various vital sign detections points surrounding the forearm of the user and is able to detect and record a respiratory rate, a pulse rate, a blood pressure measurement, and a body temperature measurement for monitoring by the user.

### CROSS-REFERENCE TO RELATED APPLICATION

The present application is a continuation in part (CIP) related to and claims priority from pending non-provisional application Ser. No. 13/815,511 filed Mar. 8, 2013 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### FIELD OF THE INVENTION

The present invention relates generally to the field of medical devices and more specifically relates to a medical device that is wearable and is used to monitor vital signs.

### DESCRIPTION OF THE RELATED ART

A medical device is an instrument, apparatus, implant, in vitro reagent, or similar related article that is used to diagnose, prevent, or treat disease or other conditions. Medical devices vary greatly in complexity and application. Simple devices such as tongue depressors, medical thermometers, and disposable gloves to advanced devices such as computers which assist in the conduct of medical testing, implants, and prostheses may fall into this category. Currently there are several medical devices that can be used however some may be bulky and not easily portable. It is desirable that medical devices be portable yet accurate as to the parameters that they record.

Many people in modem society use mobile phones for communication. A smart phone is a mobile phone with advanced computing capability. Smartphones typically include the features of a computer with those of another popular consumer device, such as a personal digital assistant (PDA), a media player, a digital camera, and/or a GPS navigation unit. Many people use cellular phones but their use may be limited to non-medical applications.

Various attempts have been made to solve the above-mentioned problems such as those found in U.S. Pub. No. 2012/0330112 to Lamego et al., U.S. Pub. No. 20110224564 to Moon et al. This art is representative of medical devices. None of the above inventions and patents, taken either singly or in combination, is seen to describe the invention as claimed.

Ideally, an omnisign medical device system should provide an effective means to monitor four vital signs of a user-wearer and, yet would operate reliably and be manufactured at a modest expense. Thus, a need exists for a reliable omnisign medical device to avoid the above-mentioned problems.

### BRIEF SUMMARY OF THE INVENTION

In view of the foregoing disadvantages inherent in the known medical device art, the present invention provides a novel omnisign medical device. The general purpose of the present invention, which will be described subsequently in greater detail is to provide a means to monitor four vital signs of a user-wearer.

An omnisign medical device system for use with a mobile communication device via an omnisign mobile software application is disclosed herein comprising a vital sign detection assembly. The vital sign detection assembly may comprise an arm cuff air bladder comprising a pressure sensor having a 0 to 5 PSI gauge pressure sensor with a sensitivity of 5.0 mV/V/FSS, a temperature sensor comprising a thermistor which may be able to detect 0 C to 50 C, a first-acoustic sensor, a second-acoustic sensor, a plastic enclosure comprising a rocker switch for activating and deactivating the vital sign detection assembly, a forearm-attacher comprising a hook-and-loop adjustable attacher, and a vital sign display device which may comprise a memory storage drive for reading and writing data file to a micro SD card.

The plastic enclosure in preferred embodiments may integrally comprise a microcontroller printed circuit board comprising a low energy 32 bit microcontroller comprising a plurality of vital sign detecting algorithms, a wireless transmitter module, and a power source comprising a rechargeable lithium-ion battery. The vital sign display device may comprise a display-device-housing, a display-device-assembly, an LCD display screen, and a display-device-assembly power supply. The omnisign medical device system may further comprise an omnisign mobile software application which may enable a user-supported mobile communication device to interact with the omnisign medical device system.

The vital sign detection assembly and the vital sign display device may comprise in functional combination the omnisign medical device system. The microcontroller printed circuit board, the wireless transmitter module, and the power source may be connected via a plurality of circuitry wires. The microcontroller printed circuit board controls the vital sign detection assembly, and the power source provides operating power to the vital sign detection assembly. The plastic enclosure may comprise a USB port such that the omnisign medical device system is able to communicate with the vital sign display device via a USB cable.

The temperature sensor, first-acoustic sensor, and second-acoustic sensor may be located about an exterior of the plastic enclosure and connected to the vital sign detection assembly. The plastic enclosure may be securely mounted to the arm cuff air bladder and the arm cuff air bladder may be securable about a forearm of a user via forearm-attacher during an ‘in-use’ condition such that the temperature sensor, the first-acoustic sensor, and the second-acoustic sensor are in touch-contact with the vital sign detection area of a user. The arm cuff air bladder may comprise a pneumatic pump comprising a 30 Vdc air pump, and at least one valve working in functional combination to measure a blood pressure of a user (wearer). The first-acoustic sensor may be structured and arranged to detect a heart rate of a user by detecting a heartbeat from within a chest cavity of the chest of the user. The second-acoustic sensor may be structured and arranged to detect a respiratory rate of the user by detecting a breathing pattern produced by a pair of lungs of the user from within the chest cavity. The temperature sensor may be placeable against an underarm of the user for detecting a body temperature of the user.

The display-device-housing, display-device-assembly, LCD display screen, and display-device-assembly power supply may comprise in functional combination the vital sign display device. The display-device-assembly may be securely mounted inside a hollow confine of display-device-housing and may be powered by the display-device-assembly power supply. The display-device-assembly may comprise a wireless transceiver module for wirelessly receiving data from the wireless transmitter module of the vital sign detection assembly. The LCD display screen may be installed to a front of the display-device-housing. It should be appreciated that the omnisign medical device system may be structured and arranged to communicate the blood pressure, the heart rate, the respiratory rate, and the body temperature of the user to the vital sign display device for displaying the vital signs of the user in real-time on the LCD display screen, as well as a user-preferred communication device.

The omnisign medical device system may further comprise a kit including the vital sign detection assembly, the vital sign display device, and a set of user instructions. The vital sign detection assembly may comprise the arm cuff air bladder, the temperature sensor, the first-acoustic sensor, the second-acoustic sensor, the plastic enclosure, and the forearm-attacher. The vital sign display device may comprise the display-device-housing, the display-device-assembly, the LCD display screen, and the display-device-assembly power supply.

A method of using the omnisign medical device system may comprise the steps of placing a vital sign detection assembly around a forearm of a user, adjusting a size of an arm cuff air bladder to fit the forearm of the user, positioning a temperature sensor, a first-acoustic sensor, and a second-acoustic, in touch-contact with the vital sign area of the user, and monitoring vital sign of user via a vital sign display device.

The present invention holds significant improvements and serves as an omnisign medical device system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, omnisign medical device, constructed and operative according to the teachings of the present invention.

FIG. 1 shows a perspective view illustrating an omnisign medical device during an ‘in-use’ condition on a forearm of a user measuring various vital signs of the user according to an embodiment of the present invention.

FIG. 2A is a perspective view illustrating a front of the omnisign medical device showing a first-acoustic sensor, a second-acoustic sensor, and a plastic enclosure according to an embodiment of the present invention of FIG. 1.

FIG. 2B is a top-perspective view illustrating the omnisign medical device showing the first-acoustic sensor, the second-acoustic sensor, and the acoustic sensor housing according to an embodiment of the present invention of FIG. 2A.

FIG. 3 is a perspective view illustrating the omnisign medical device showing an arm cuff air bladder comprising a pressure sensor and a power source according to an embodiment of the present invention of FIGS. 1 & 2A.

FIG. 4 is a perspective view illustrating the omnisign medical device showing a microcontroller printed circuit board comprising a plurality of vital sign detecting algorithms, a wireless transmitter module, a rechargeable lithium-ion battery power source, and a memory storage drive for reading and writing data file to a micro SD card according to an embodiment of the present invention of FIGS. 1-3.

FIG. 5 is a perspective view illustrating the omnisign medical device wirelessly communicating with a vital sign display device comprising a user-provided communication device such as a laptop computer, a smartphone, or a tablet computer, according to an embodiment of the present invention of FIGS. 1-4.

FIG. 6 illustrates a method of use for the omnisign medical device.

The various embodiments of the present invention will hereinafter be described in conjunction with the appended drawings, wherein like designations denote like elements.

### DETAILED DESCRIPTION

As discussed above, embodiments of the present invention relate to a medical device and more particularly to an omnisign medical device as used to improve the monitoring of vital signs.

Generally speaking, the system disclosed herein is designed to measure up to four vital signs of the human body with one instrument linked wirelessly to an Android/iPhone Tablet or Smartphone application used by health care professionals. These vital signs include blood pressure, heart pulse rate, body temperature and respiratory rate. The instrument is housed in a plastic enclosure (or other suitable material) with an attached arm cuff air bladder which is wrapped around the upper left arm and held in place with a hook and loop connection. Within the enclosure are temperature and acoustic sensors, pneumatic pump and valves along with the microcontroller printed circuit board and rechargeable battery. The instrument is positioned over the arm and against the inner left chest while being worn for measurements to take place.

The system is controlled by a high performance, low energy microcontroller. The present invention is preferably powered by a rechargeable lithium-ion battery and is recharged by a supplied wall power supply or through an available USB port. Communications with the Android/iPhone Tablet or Smartphone are carried out by a BLUETOOTH® transceiver or the like.

Blood Pressure may be measured by inflating the air bladder and slowly releasing and measuring the pressure until the diastolic and systolic points are detected by the acoustic sensor located over the Brachial Artery of the arm. The acoustic sensor detects the sounds from within the arm and sends the signals through a band pass filter/amplifier to an analog to digital converter to be processed by the algorithms programmed into the microcontroller for determining the blood flow characteristics that indicate the diastolic and systolic pressure points. At these two points, the air bladder pressure sensor is read to indicate the Blood Pressure.

Heart Rate and Respiratory Rate are measured using a second acoustic sensor; this sensor located and placed against the left side of the chest under the armpit. Again, the sensor detects sounds, this time from within the chest cavity, and sends the signals through a band pass filter/amplifier to an analog to digital converter to be processed by the algorithms programmed into the microcontroller for determining the heart pulse rate and respiratory rate.

Body temperature may be measured by a thermistor placed against the underarm and processed by the microcontroller. All measurements will be transmitted via BLUETOOTH® to the Android/iPhone® device for display to the user/operator. These measurements can be recorded locally on a MicroSD card and later downloaded to a wireless network for further processing.

The heart of the Omnisign system is a high performance, low energy 32 bit microcontroller from Silabs, #EFM32LG230F256-QFN64. This is a highly integrated MCU tailored around a Cortex-M3 core. It operates at 48.000 MHz and incorporates 256 Kbytes flash memory with 32 Kbytes of SRAM. Peripherals include 8 channels of 12 bit 1 Msamples/sec ADC, 1×12 bit 500 ksamples/sec DAC, 2×Analog Comparators, Supply Voltage Comparator, 2×I2C buses, 3×SPI buses, 2×Low Energy UARTs as well as GPIO ports. The MCU is factory programmed via the ICSP connector.

The system is powered by a rechargeable 3.7V, 2600 mAh Li-Ion battery in preferred embodiments. The battery supplies power to a Buck-Boost switch mode regulator that converts the battery voltage to +4.3V that is used to supply three separate LDO regulators. These LDO's are used to provide low noise power to separate circuits across the system. Each LDO supplies +3.3V at less than 1 amp each, one for the digital circuits, one for the analog circuits and one for peripherals. The Buck-Boost is also used to operate at battery input voltages down to +2.4V, which allows the system to use up as much of the battery power as possible before needing to be recharged. The system is recharged from either a wall power supply or an available USB port through the Micro B USB receptacle. This port may also be used to deliver power for the system while recharging the battery. A Power ON rocker switch controls a MOSFET power switch that allows current to flow to the input of the Buck-Boost and therefore powers on the system. An LED indicates when the battery is charging and another LED indicates when the Power is ON to the system.

The system uses several sensors to detect the body's vital signs. Two acoustic sensors are used to listen for sounds from within the body, one for the blood pressure and one for the heart pulse rate and respiratory rate. A thermistor is used to detect body temperature. The acoustic sensors' output are feed through band pass filters to block unwanted frequencies from entering the amplifier circuits. After being amplified by chopper stabilized op-amps, the signals then enter separate 12 bit, 1 Msamples/sec ADC channels of the MCU. Through use of FFT digital signal processing, the wanted sounds are identified and rates are determined.

For blood pressure measurements, the pressure of the arm cuff air bladder is sensed by an onboard 0 to 5 psi gauge pressure sensor. The sensor's output is feed directly to a differential input, 24 bit Delta-Sigma ADC. Both the sensor and the ADC use the +3.3 VA power rail for their references. The MCU uses a SPI bus to interface with the ADC.

The body temperature is detected by a 0 C to 50 C thermistor. A voltage divider network is formed by a 10 K ohms precision resistor and the thermistor and the voltage between the two is buffered by a chopper stabilized op-amp and feed to a channel of the ADC internal of the MCU for processing.

The air pressure needed to inflate the arm cuff air bladder may be provided by a 3.0 Vdc air pump. This pump is controlled by a GPIO port of the MCU and driven by an inductive back EMF protected transistor. The air bladder pressure, once the blood pressure has been detected, is exhausted through a 3.0 Vdc solenoid valve which is also controlled by a GPIO port of the MCU and a similar protected transistor. Both outputs are over-current protected by PTC type resettable fuses.

The user/operator interfaces with the system through the application program running on either a Tablet or Smartphone. This device communicates with the sensor controller device via BLUETOOTH®. A 2.4 GHz Bluetooth transceiver is located on board and is connected to the MCU through a UART bus interface. LEDs are connected to the BLUETOOTH® module to indicate activity.

The power supplies begin with the source of power which is a 3.7V 2600 mAh Lithium-Ion rechargeable battery. This battery is Varta #56627-201-013 and is a standard size 18650 cylinder. The battery has its own Overcharge Detection@4.300V, Overdischarge Detection@2.400V (with a 76.8 to 115.2 msec delay, resumed by removing load) and also Overcurrent Detection of 6 to 8 A (with a 9.6 to 14.4 msec delay). It has an internal resistance of approximately 150 m ohms

The system includes a recharging controller U10,that limits the charge current to 500 ma and shuts off when the battery voltage is sensed at 4.2V. The charge port is a Micro B USB receptacle. A wall power supply of +5.0V is used to provide charging current or an available USB port may also be used. The incoming power from the charge port is RF filtered then overvoltage and overcurrent as well as reversed polarity protected by L18, D3, F4 & Q4 (P-channel MOSFET). This allows a minimum of voltage loss at operating current levels. The system may operate from either the battery or charge port as Q6 (an Ideal Diode Switch) selects which source to use to power the system. A rocker switch connected to J10 controls another P-channel MOSFET, Q5, which serves as the POWER ON switch. Both Q4 and Q5 have internal overvoltage protection.

When the power switch is ON, Q5 is switched ON, allowing current to flow with a minimum of voltage loss, to the input of U7, a Buck-Boost switch mode regulator programmed by resistor divider network, R21 & R17, to produce +4.3V at its output. The internal error amplifier is type III compensated with a network of R24, C50, C49, C45 and R23. C47 is used as an output storage filter.

To provide isolation of analog, digital and high current, high inductive peripherals circuits, three separate LDO's are incorporated to regulate the +4.3V output of the Buck-Boost to +3.3V for each of the circuits. These are internally protected, fixed output, 1 Amp linear regulators with a less than 500 mV dropout voltage rating. By limiting the input voltage to +4.3V, less heat is needed to dissipate from each LDO, making the supply more efficient, therefore making better use of the battery power source.

As stated previously, the Omnisign system uses a high performance, low energy 32 bit microcontroller from Silabs, #EFM32LG230F256-QFN64 as its central controller. This is a highly integrated MCU tailored around a Cortex-M3 core. It operates at 48.000 MHz and incorporates 256 Kbytes flash memory with 32 Kbytes of SRAM. Peripherals include 8 channels of 12 bit 1 Msamples/sec ADC, 1×12 bit 500 ksamples/sec DAC, 2×Analog Comparators, Supply Voltage Comparator, 2×I2C buses, 3×SPI buses, 2×Low Energy UARTs as well as GPIO ports. The MCU is factory programmed via the ICSP connector J1. The MCU operates from the +3.3 VD power rail.

Communications with a Graphical User's Interface (Tablet/Smartphone App), is performed with a BLUETOOTH® transceiver which is interfaced with the MCU through its LEUART_{—}0 port. Included with this I/F is a GPIO pin configured to be used as the BT transceiver Reset signal.

Interface with the 24 bit Delta Sigma Differential ADC, which senses the onboard Pressure Sensor output, is enabled via the SPI_{—}2 bus. ‘PSI CS #’ is the chip select for this ADC I/F. The acoustic sensors for the blood pressure, heart pulse and respiratory rate, are conditioned and then sampled by the internal 12 bit ADC on channels 0 & 1. Body temperature is buffered and sampled by channel 2 of the internal ADC. This ADC is referenced to either the external +1.5 Vbias on channel 6 or the internal +1.25 Vref voltages. Channel 7 is used as −Vref.

Peripheral outputs, exhaust valve, bleed valve and air pump are controlled by GPIO. Supply voltage is monitored by analog comparator ACMP1_CH0 and compared to an internal 2.5V reference.

The arm cuff air bladder pressure is sensed by an on board, temperature compensated pressure sensor such as those made by Honeywell. This is a 0 to 5 psi gauge pressure sensor with a sensitivity of 5.0 mV/V/FSS. It is excited by the +3.3 VA voltage rail that is also used as the reference voltage to the 24 bit, Delta Sigma Differential Input ADC in which the sensor is feed into. The ADC interfaces with the MCU via its SPI bus.

The body temperature is sensed by a Honeywell Series 194, 10 Kohm, 0 to 50 degree Celsius thermistor. The thermistor has an accuracy of +/−0.2 degrees C. The thermistor is the bottom half of a voltage divider circuit formed with R31, a 10 Kohm precision resistor. The voltage, taken from the center of the divider network, is buffered by U5:A, a voltage follower with unity gain, to feed the input of Channel 2 of the MCU's internal ADC. The un-buffered input of U5:A is protected from extending past either power rail by D8, a dual Schottky Diode package.

Sounds from inside the body are detected by the use of acoustic sensors strategically placed on the body so as to sense the desired functions. For the blood pressure, a low frequency sensitive (<10 Hz), MEMS acoustic sensor is feed into the “A” channel signal conditioning circuit while the sensor for the heart pulse rate and respiratory rate, is an electret condensing element and feed into Channel “B”. Channel “A” is setup to provide operating power to the MEMS sensor from the +3.3 VA voltage rail. Channel “B” is setup to provide bias voltage to the Electret Condensing element from a +1.5 Vbias rail. This is regulated from a precision voltage reference U4, up to 25 ma.

Both input channels from there are RF filtered and AC coupled through a 2.2 uf capacitor to the non-inverting inputs of chopper stabilized, 3 MHz dual opamps. The opamps form a Multi Feedback Bandpass Filter/Amplifier circuit centered around 100 Hz and a gain of 1000 with a damping ratio of 1.00 and a Q of 0.498. A second Low Pass Filter is comprised of a 0.15 uf capacitor in parallel with a series RC network of a 4.99 ohm resistor and a 1.0 uf capacitor. This filter has a −3db frequency of 32 KHz. The outputs of the conditioning circuits are feed into their respective channels of the MCU's internal ADC for digital signal processing.

The arm cuff air bladder is pressurized by a 3.0V mini diaphragm air pump. The pump is powered by the +3V power rail and driven by the self-protected MOSFET transistor package Q1 which is controlled by a GPIO of the MCU. The output is overcurrent protected by F1, a PTC resettable 1A fuse.

The air bladder must be deflated after measurement is complete. This is exhausted by a solenoid valve powered by the +3V power rail and driven by a self-protected MOSFET transistor package Q3 which is controlled by a GPIO of the MCU. The output is overcurrent protected by F3, a PTC resettable 1A fuse. The pressure in the air bladder must be released at a slow rate for accurate measurement. This is released by a solenoid valve powered by the +3V power rail and driven by a self-protected MOSFET transistor package Q2 which is controlled by a GPIO of the MCU. The output is overcurrent protected by F2, a PTC resettable 1A fuse. Communications with the GUI device is via BLUETOOTH® v3.0 transceiver U2. This is a 2.4 GHz BT class-2 module with integrated chip antenna. Interface to the MCU is through its UART bus. It uses an “AT2” command set and is fully FCC and BLUETOOTH® qualified. It is powered by the +3.3 VA power rail. The module includes a Cortex-M3 microcontroller with its own firmware stack.

The current GUI is a custom Android application program operating on a Kocaso M776H 7″ display Tablet. Included is an ARM dual-core CORTEX A9 OMAP 4 running at 1.2 GHz with 8 GB Flash Memory and 1 GB RAM Memory.

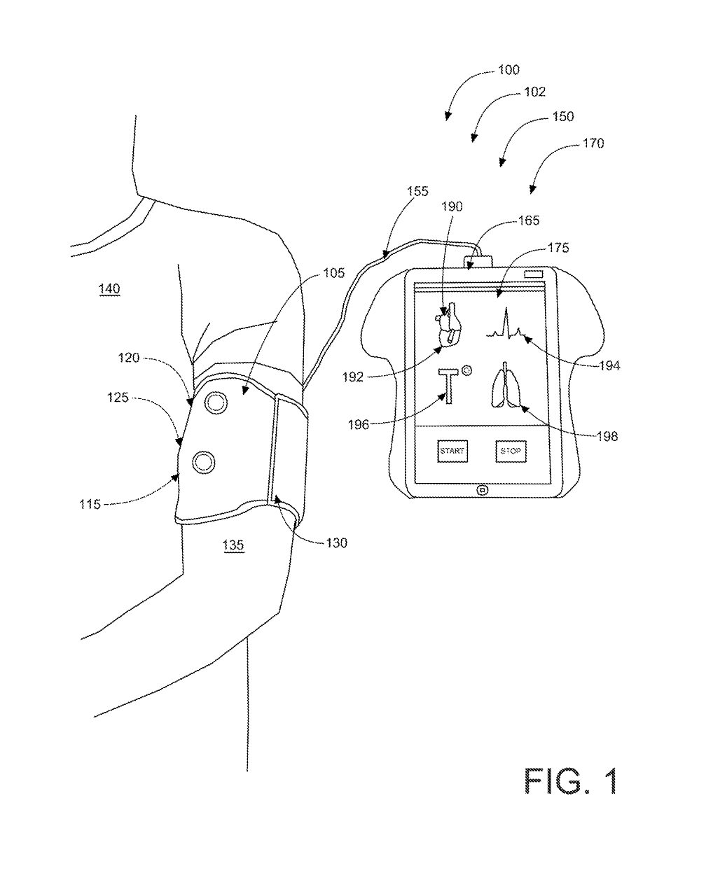

Referring now to the drawings more specifically by numerals of reference there is shown in FIG. 1, omnisign medical device systems 100 during ‘in-use’ condition 150 according to an embodiment of the present invention. Generally speaking, omnisign medical device systems 100 may be designed to measure up to four vital signs of the human body with one instrument linked wirelessly to an Android/iPhone Tablet or Smartphone application used by health care professionals. Omnisign medical device system 100 may comprise vital sign detection assembly 102 and vital sign display device 160 working in functional combination for providing user 140 with real-time awareness of at least one vital sign (preferably four vital signs). Vital sign(s) may be displayed on vital sign display device 160 and may include blood pressure 192, heart rate 194, respiratory rate 198, and body temperature 196. Omnisign medical device systems 100 for use with a mobile communication device via omnisign software application 580 may further comprise vital sign detection assembly 102, vital sign display device 160 and omnisign software application 580. Vital sign display device 160 may comprise a memory storage drive for reading and writing at least one data file to a micro SD card.

In continuing to refer to FIG. 1, vital sign detection assembly 102 may comprise arm cuff air bladder 105, temperature sensor 115, first-acoustic sensor 120, second-acoustic sensor 125, plastic enclosure 110, and forearm-attacher 130. The instrument may be housed in plastic enclosure 110 with an attached arm cuff air bladder 105 which may be wrapped around forearm 135 and held in place with a hook and loop connection or other suitable fastening means. Within the enclosure may be temperature sensor 115, first-acoustic sensor 120, second-acoustic sensor 125, pneumatic pump 310 and valves along with the microcontroller printed circuit board and rechargeable battery. Arm cuff air bladder 105 may comprise a pressure sensor having 0 to 5 PSI gauge, the pressure sensor may have a sensitivity of 5.0 mV/V/FSS. Temperature sensor 115 may comprise a thermistor which may be able to detect 0 C to 50 C.

In still referring to FIG. 1, plastic enclosure 110 may comprise rocker switch 330 for activating and deactivating vital sign detection assembly 102. Plastic enclosure 110 may integrally comprise microcontroller printed circuit board which may comprise low energy 32 bit microcontroller 210, wireless transmitter module 215, and power source 220 which may comprise a rechargeable lithium-ion battery. The instrument may be positioned over forearm 135 and against the inner left chest while being worn for measurements to take place. During ‘in-use’ condition 150, user 140 may place arm cuff air bladder 105 around forearm 135 in such a manner that temperature sensor 115, first-acoustic sensor 120, and second-acoustic sensor 125 are in touch-contact with different vital sign detection areas around forearm 135 and side-chest-area of user 140. Vital sign detection areas may include a brachial artery, side-chest, and forearm 135. Vital sign detection assembly 102 may then communicate vital signs of user 140 to vital sign display device 160 via wireless signal 525.

Vital sign display device 160 of omnisign medical device systems 100 may comprise display-device-housing 165, display-device-assembly 170, LCD display screen 175, and display-device-assembly power supply 180. Display-device-assembly 170 may comprise display-device-housing 165, LCD display screen 175, and display-device-assembly power supply 180. Display-device-assembly 170 may be securely mounted inside a hollow confine of display-device-housing 165, and display-device-assembly 170 may further be powered by display-device-assembly power supply 180. LCD display screen 175 may be installed to a front of display-device-housing 165, and omnisign medical device systems 100 may be structured and arranged to communicate blood pressure 192, heart rate 194, respiratory rate 198, and body temperature 196 of user 140 to vital sign display device 160 for displaying vital sign of user 140 in real-time on LCD display screen 175.

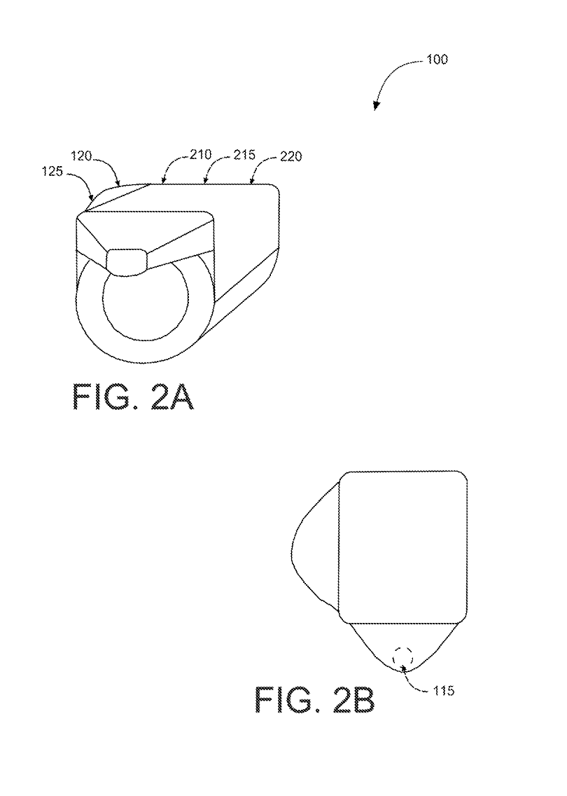

Referring now to FIGS. 2A-2B, omnisign medical device systems 100 may comprise first-acoustic sensor 120, second-acoustic sensor 125, and plastic enclosure 110 comprising rocker switch 330 for activating and deactivating vital sign detection assembly 102. Plastic enclosure 110 may integrally comprise microcontroller printed circuit board 210, wireless transmitter module 215, and power source 220. Microcontroller printed circuit board 210 of vital sign detection assembly 102 may comprise a low energy 32 bit microcontroller and a plurality of vital sign detecting algorithms may be programmed into the low energy 32 bit microcontroller. Power source 220 for powering vital sign detection assembly 102 may comprise a rechargeable lithium-ion battery. Temperature sensor 115 may comprise thermistor, which may be able to detect temperatures from 0 C to 50 C. Microcontroller printed circuit board 210, wireless transmitter module 215, and power source 220 may be connected via a plurality of circuitry wires. Microcontroller printed circuit board 210 may control vital sign detection assembly 102 and power source 220 may provide operating power to vital sign detection assembly 102.

In continuing to refer to FIGS. 2A-2B, low energy 32 bit microcontroller printed circuit board 210 may comprise a plurality of vital sign detecting algorithms for controlling vital sign detection assembly 102 of omnisign medical device systems 100 during ‘in-use’ condition 150. The plurality of vital sign detecting algorithms may be able to detect at least one vital sign of user 140, then process and send the information via wireless transmitter module 215 to vital sign display device 160. Vital sign display device 160 may comprise display-device-housing 165, display-device-assembly 170, LCD display screen 175, and display-device-assembly power supply 180. Microcontroller printed circuit board 210, wireless transmitter module 215 and power source 220 may be connected via plurality of circuitry wires. Microcontroller printed circuit board 210 may control vital sign detection assembly 102. Power source 220 may provide operating power to vital sign detection assembly 102.

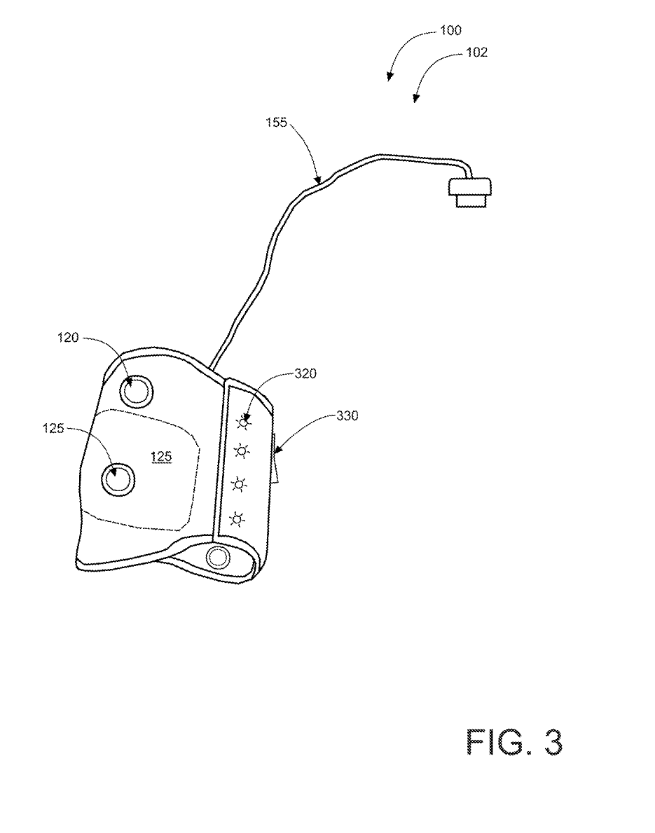

Referring now to FIG. 3, vital sign detection assembly 102 of omnisign medical device systems 100 may comprise arm cuff air bladder 105, forearm-attacher 130, temperature sensor 115, first-acoustic sensor 120, second-acoustic sensor 125, plastic enclosure 110, USB cable 155, and LED indicator lights 320. Arm cuff air bladder 105 may comprise pneumatic pump 310 and valve working in functional combination to measure blood pressure 192 of user 140. Arm cuff air bladder 105 may be securable about forearm 135 of user 140 via forearm-attacher 130 such that temperature sensor 115, first-acoustic sensor 120, and second-acoustic sensor 125 are in touch-contact with vital sign detection area of user 140. Forearm-attacher 130 may comprise a hook-and-loop adjustable attacher, such as VELCRO® (hook and loop fastening means), thus enabling arm cuff air bladder 105 to fit forearms of different sizes.

In continuing to refer to FIG. 3, temperature sensor 115, first-acoustic sensor 120, and second-acoustic sensor 125 may be located about an exterior of plastic enclosure 110 and connected to vital sign detection assembly 102. Temperature sensor 115 may comprise a thermistor which may be used to detect body temperature 196. Temperature sensor 115 comprising the thermistor may be placed against and between a side-chest area and an underarm of user 140 which may be optimal for detecting body temperature 196 of user 140. Temperature sensor 115 may measure body temperature 196 of user 140 and communicate body temperature 196 to microcontroller printed circuit board 210. All measurements may be transmitted directly or via Bluetooth to vital sign display device 160 for display to user 140. These measurements may be recorded locally on a MicroSD card and later downloaded to a wireless network for further processing.

In continuing to refer to FIG. 3, first-acoustic sensor 120 may be structured and arranged to detect heart rate 194 of user 140 by detecting a heartbeat from within a chest cavity of a chest of user 140. First-acoustic sensor 120 may further be structured and arranged to measure blood pressure 192. Blood pressure 192 may be measured by inflating arm cuff air bladder 105 and slowly releasing and measuring the pressure until the diastolic and systolic points are detected by first-acoustic sensor 120 located over the brachial artery of forearm 135 of user 140. First-acoustic sensor 120 may detect the sounds from within forearm 135 and send the signals through a band pass filter/amplifier to an analog to digital converter to be processed by the algorithms programmed into microcontroller printed circuit board 210 for determining the blood flow characteristics that indicate the diastolic and systolic pressure points. At these two points, vital sign information from arm cuff air bladder 105 may be displayed on vital sign display device 160 and read to indicate blood pressure 192. Second-acoustic sensor 125 may be structured and arranged to detect respiratory rate 198 of user 140 by detecting a breathing pattern produced by lungs of user 140 from within the chest cavity. Second-acoustic sensor 125 may be located and placed against the left side of the chest of user 140 under the armpit. Second-acoustic sensor 125 may detect sounds from within the chest cavity, and may send the signals through a band pass filter/amplifier to an analog to digital converter to be processed by the algorithms programmed into microcontroller printed circuit board 210 for determining heart rate 194 and respiratory rate 198.

In continuing to refer to FIG. 3, plastic enclosure 110 may comprise a plurality of LED indicator lights 320 powered by power source 220 for indicating wireless signal 525 transmission of wireless transmitter module 215. Plastic enclosure 110 may be securely mounted to arm cuff air bladder 105. Plastic enclosure 110 may further comprise rocker switch 330 for activating and deactivating vital sign detection assembly 102. LED indicator lights 320 may comprise small lights that may be integral to plastic enclosure 110, and may light up during ‘in-use’ condition 150 of omnisign medical device systems 100. Plastic enclosure 110 may further comprise a USB port allowing omnisign medical device systems 100 to communicate with vital sign display device 160 via USB cable 155. USB cable 155 may comprise a length of cable attached on one end to omnisign medical device systems 100 and on the other end may comprise a USB connector for electronically connecting to a USB port on vital sign display device 160.

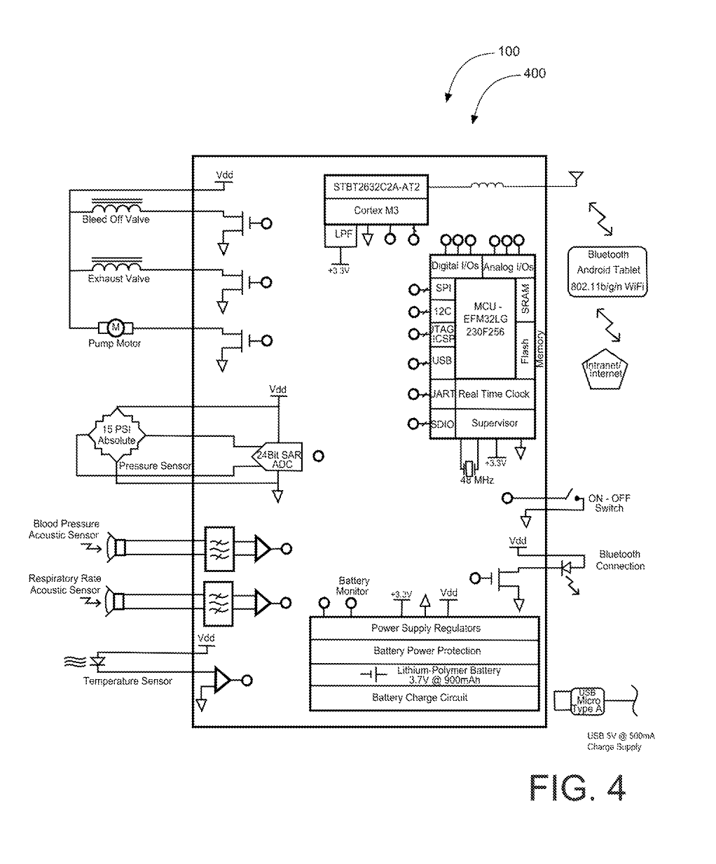

Referring now to FIG. 4, communication diagram 400 of omnisign medical device systems 100 illustrates an electrical flow for omnisign medical device systems 100 in a preferred embodiment of the present invention. Communication diagram 400 may comprise microcontroller printed circuit board 210 which may comprise a low energy 32 bit microcontroller. The low energy 32 bit microcontroller may comprise a plurality of vital sign detecting algorithms, wireless transmitter module 215, power source 220 comprising a rechargeable lithium-ion battery, and a memory storage drive for reading and writing data file to a micro SD card.

In continuing to refer to FIG. 4, communication diagram 400 in physical representation may comprise microcontroller printed circuit board 210 located within vital sign detection assembly 102. During ‘in-use’ condition 150, arm cuff air bladder 105 may be powered by pneumatic pump 310 comprising 30 Vdc air pump. Arm cuff air bladder 105 may comprise pressure sensor having 0 to 5 PSI gauge pressure sensor with a sensitivity of 5.0 mV/V/FS S. Microcontroller printed circuit board 210 of vital sign detection assembly 102 may further comprise a bandpass filter/amplifier circuit centered around 100 Hz and having a gain of 1000 with a damping ratio of 1.0 and a Q of 0.498. Vital sign detection assembly 102 may further comprise a second low pass filter, the second low pass filter comprising a 0.15 capacitor. An acoustic sensor may be able to detect diastolic and systolic points of user 140 and communicate diastolic and systolic points to bandpass filter/amplifier circuit to an analog-to-digital converter.

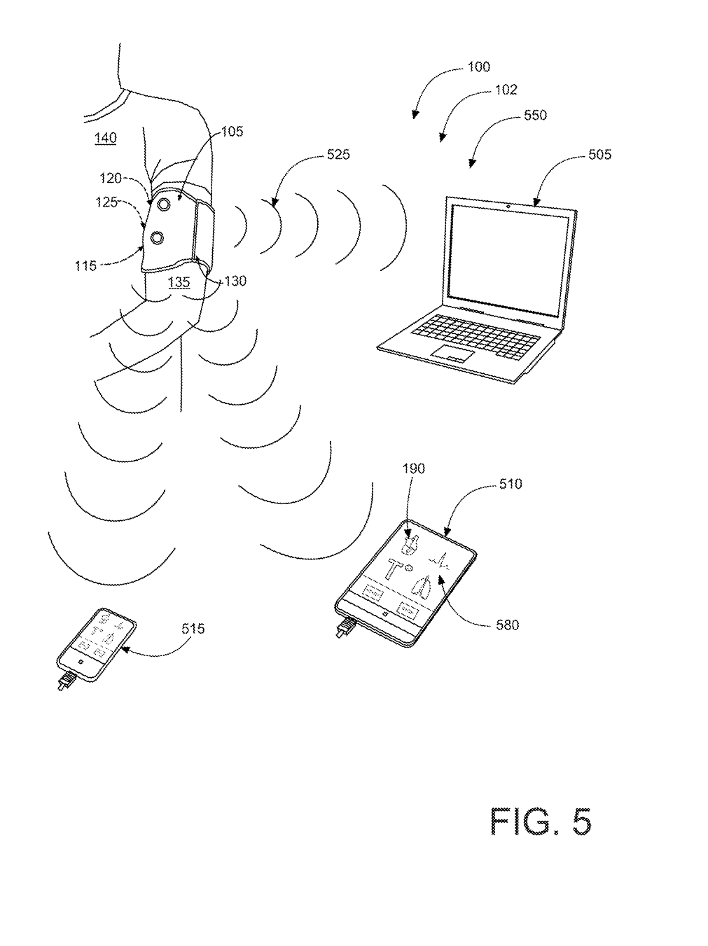

Referring now to FIG. 5, showing omnisign medical device systems 100 wirelessly communicating with vital sign display device 160 comprising a user-provided communication device such as laptop 505, tablet 510, or smartphone 515 according to an embodiment of the present invention. As shown, vital sign display device 160 comprising a wireless communication interface may receive wireless signal 525 from wireless transmission from wireless transmitter module 215 of vital sign detection assembly 102 for displaying information relating to at least one vital sign of user 140 on a display screen of the user-provided communication device.

In continuing to refer to FIG. 5, vital sign detection assembly 102 is shown comprising arm cuff air bladder 105, temperature sensor 115, first-acoustic sensor 120, second-acoustic sensor 125, plastic enclosure 110, and forearm-attacher 130. During ‘in-use’ condition 550, user 140 may place arm cuff air bladder 105 around forearm 135 in such a manner that temperature sensor 115, first-acoustic sensor 120, and second-acoustic sensor 125 are in touch-contact with different vital sign detection areas around forearm 135 and side-chest-area of user 140. Vital sign detection assembly 102 may then wirelessly communicate vital signs blood pressure 192, heart rate 194, respiratory rate 198, and body temperature 196 of user 140 to vital sign display device 160 for displaying vital sign(s) of user 140 in real-time on LCD display screen 175.

In continuing to refer to FIG. 5, omnisign medical device systems 100 may comprise a memory storage drive for reading and writing a data file to a micro SD card, which may then be uploaded to a user-supported mobile communication device. Omnisign medical device systems 100 may further comprise omnisign software application 580. Omnisign software application 580 may enable the user-supported mobile communication device to interact with omnisign medical device systems 100. The user-supported mobile communication device may comprise smartphone 515 and alternatively tablet 510 or laptop 505. Omnisign medical device systems 100 may comprise display-device-assembly 170. Display-device-assembly 170 may comprise a wireless transceiver module for wirelessly receiving a byte of data from wireless transmitter module 215 of vital sign detection assembly 102. Vital sign detection assembly 102 and vital sign display device 160 may comprise in functional combination omnisign medical device systems 100.

In continuing to refer to FIG. 5, display-device-housing 165, display-device-assembly 170, LCD display screen 175, and display-device-assembly power supply 180 may comprise in functional combination vital sign display device 160. Display-device-housing 165 may comprise smartphone 515 and alternatively tablet 510 or laptop 505. Display-device-assembly 170 may comprise wireless transceiver module for wirelessly receiving at least one byte of data from wireless transmitter module 215 of vital sign detection assembly 102. Display-device-assembly 170 may be securely mounted inside a hollow confine of display-device-housing 165. Display-device-assembly 170 may be powered by display-device-assembly power supply 180. LCD display screen 175 may be installed to front of display-device-housing 165. Omnisign medical device systems 100 may be structured and arranged to communicate blood pressure 192, heart rate 194, respiratory rate 198, and body temperature 196 of user 140 to vital sign display device 160 for displaying at least one vital sign of user 140 in real-time on LCD display screen 175.

Referring generally now to FIGS. 1-5, showing omnisign medical device systems 100; Omnisign medical device systems 100 may be sold as a kit comprising the following parts: vital sign detection assembly 102; vital sign display device 160; and at least one set of user instructions. The kit has instructions such that functional relationships are detailed in relation to the structure of the invention (such that the invention can be used, maintained, or the like in a preferred manner). Omnisign medical device systems 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different monitoring/communication/fastening combinations, parts may be sold separately, etc., may be sufficient.

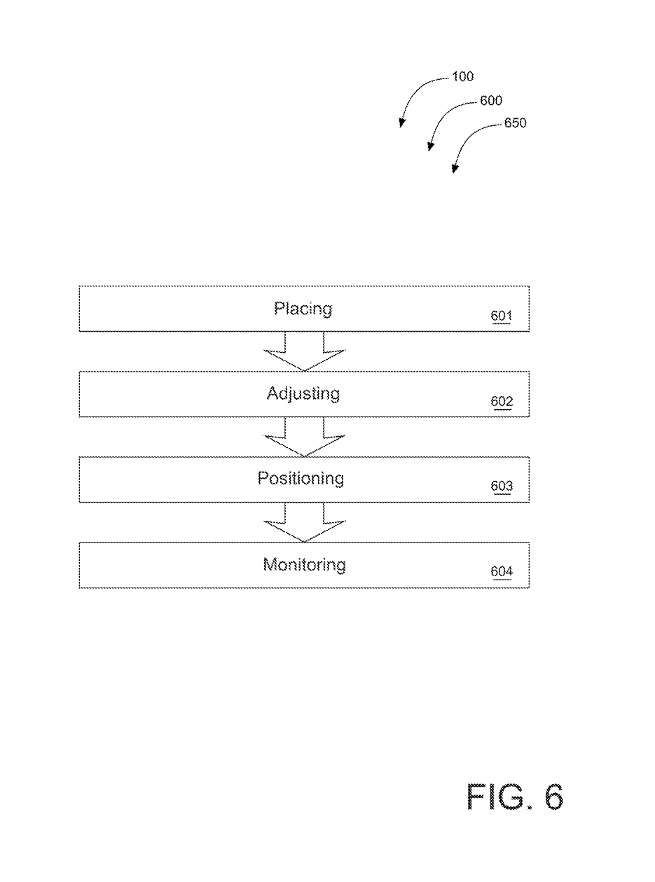

Referring now to FIG. 6, illustrating method of use 600 for omnisign medical device systems 100 and may comprise the steps of: step one 601, placing a vital sign detection assembly around a forearm of a user, step two 602, adjusting a size of arm cuff air bladder 105 to fit forearm 135 of user 140, step three 603 positioning temperature sensor 115, first-acoustic sensor 120, and second-acoustic 125, in touch-contact with the vital sign area of user 140, and step four 604, monitoring a vital sign of user 140 via vital sign display device 160.

It should be noted that the steps described in the method of use can be carried out in many different orders according to user preference. The use of “step of” should not be interpreted as “step for”, in the claims herein and is not intended to invoke the provisions of 35 U.S.C. §112, ¶6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. An omnisign medical device system comprising:
a vital sign detection assembly comprising;
an arm cuff air bladder;
a temperature sensor;
a first-acoustic sensor; and
a second-acoustic sensor;
a plastic enclosure, said plastic enclosure integrally comprising;
a microcontroller printed circuit board;
a wireless transmitter module; and
at least one power source; and
a forearm-attacher; and
a vital sign display device, said vital sign display device comprising;
a display-device-housing;
a display-device-assembly;
a LCD display screen; and
a display-device-assembly power supply;
wherein said vital sign detection assembly and said vital sign display device comprises in functional combination said omnisign medical device system;
wherein said microcontroller printed circuit board, said wireless transmitter module, and said at least one power source are connected via a plurality of circuitry wires;
wherein said microcontroller printed circuit board controls said vital sign detection assembly;
wherein said at least one power source provides operating power to said vital sign detection assembly;
wherein said temperature sensor, said first-acoustic sensor, and said second-acoustic sensor are located about an exterior of said plastic enclosure and connected to said vital sign detection assembly;
wherein said plastic enclosure is securely mounted to said arm cuff air bladder;
wherein said arm cuff air bladder is securable about a forearm of a user via said forearm-attacher such that said temperature sensor, said first-acoustic sensor, and said second-acoustic sensor are in touch-contact with at least one vital sign detection area of said user;
wherein said arm cuff air bladder comprises a pneumatic pump and at least one valve working in functional combination to measure a blood pressure of said user;
wherein said first-acoustic sensor is structured and arranged to detect a heart rate of said user by detecting a heartbeat from within a chest cavity of said chest of said user;
wherein said second-acoustic sensor is structured and arranged to detect a respiratory rate of said user by detecting a breathing pattern produced by lungs of said user from within said chest cavity;
wherein said temperature sensor is placeable against an underarm of said user for detecting a body temperature of said user;
wherein said display-device-housing, said display-device-assembly, said LCD display screen, and said display-device-assembly power supply comprises in functional combination said vital sign display device;
wherein said display-device-assembly is securely mounted inside a hollow confine of said display-device-housing;
wherein said display-device-assembly is powered by said display-device-assembly power supply;
wherein said display-device-assembly comprises a wireless transceiver module for wirelessly receiving at least one byte of data from said wireless transmitter module of said vital sign detection assembly;
wherein said LCD display screen is installed to a front of said display-device-housing; and
wherein said omnisign medical device system is structured and arranged to communicate said blood pressure, said heart rate, said respiratory rate, and said body temperature of said user to said vital sign display device for displaying said at least one vital sign of said user in real-time on said LCD display screen.

2. The omnisign medical device system of claim 1 wherein said microcontroller printed circuit board of said vital sign detection assembly comprises a low energy 32 bit microcontroller.

3. The omnisign medical device system of claim 2 wherein a plurality of vital sign detecting algorithms are programmed into said low energy 32 bit microcontroller.

4. The omnisign medical device system of claim 3 wherein said at least one power source for powering said vital sign detection assembly comprises a rechargeable lithium-ion battery.

5. The omnisign medical device system of claim 4 wherein said temperature sensor comprises a thermistor, said thermistor able to detect 0 C to 50 C.

6. The omnisign medical device system of claim 5 wherein said arm cuff air bladder is powered by said pneumatic pump comprising a 30 Vdc air pump.

7. The omnisign medical device system of claim 6 wherein said arm cuff air bladder further comprises a pressure sensor having a 0 to 5 PSI gauge pressure sensor with a sensitivity of 5.0 mV/V/FSS.

8. The omnisign medical device system of claim 7 wherein said vital sign detection assembly further comprises a bandpass filter/amplifier circuit centered around 100 Hz and having a gain of 1000 with a damping ratio of 1.0 and a Q of 0.498.

9. The omnisign medical device system of claim 8 wherein said vital sign detection assembly further comprises a second low pass filter, said second low pass filter comprising a 0.15 of capacitor.

10. The omnisign medical device system of claim 9 wherein said first-acoustic sensor is able to detect diastolic and systolic points of said user and communicate said diastolic and systolic points to said bandpass filter/amplifier circuit to an analog to digital converter.

11. The omnisign medical device system of claim 10 wherein said plastic enclosure comprises a plurality of LED indicator lights powered by said at least one power source for indicating a wireless signal transmission of said wireless transmitter module.

12. The omnisign medical device system of claim 11 wherein said plastic enclosure comprises a rocker switch for activating and deactivating said vital sign detection assembly.

13. The omnisign medical device system of claim 12 wherein said plastic enclosure comprises a USB port such that said omnisign medical device system is able to communicate with said vital sign display device via a USB cable.

14. The omnisign medical device system of claim 13 wherein said vital sign display device further comprises a memory storage drive for reading and writing at least one data file to a micro SD card.

15. The omnisign medical device system of claim 14 further comprising an omnisign mobile software application, said omnisign mobile software application enabling a user-supported mobile communication device to interact with said omnisign medical device system.

16. The omnisign medical device system of claim 15 wherein said user-supported mobile communication device comprises a smartphone and alternatively a tablet.

17. The omnisign medical device system of claim 15 wherein said user-supported mobile communication device comprises a computer.

18. An omnisign medical device system for use with a mobile communication device via an omnisign mobile software application comprising:
a vital sign detection assembly, said vital sign detection assembly comprising;
an arm cuff air bladder comprising a pressure sensor having a 0 to 5 PSI gauge pressure sensor with a sensitivity of 5.0 mV/V/FSS;
a temperature sensor, said temperature sensor comprising a thermistor, said thermistor able to detect 0 C to 50 C;
a first-acoustic sensor;
a second-acoustic sensor; and
a plastic enclosure comprising a rocker switch for activating and deactivating said vital sign detection assembly, said plastic enclosure integrally comprising;
a microcontroller printed circuit board comprising a low energy 32 bit microcontroller, said low energy 32 bit microcontroller comprising a plurality of vital sign detecting algorithms;
a wireless transmitter module; and
at least one power source comprising a rechargeable lithium-ion battery; and
a forearm-attacher comprising a hook-and-loop adjustable attacher; and
a vital sign display device comprising a memory storage drive for reading and writing at least one data file to a micro SD card, said vital sign display device comprising;
a display-device-housing;
a display-device-assembly;
a LCD display screen; and
a display-device-assembly power supply; and
an omnisign mobile software application, said omnisign mobile software application enabling a user-supported mobile communication device to interact with said omnisign medical device system;
wherein said vital sign detection assembly and said vital sign display device comprises in functional combination said omnisign medical device system;
wherein said microcontroller printed circuit board, said wireless transmitter module, and said at least one power source are connected via a plurality of circuitry wires;
wherein said microcontroller printed circuit board controls said vital sign detection assembly;
wherein said at least one power source provides operating power to said vital sign detection assembly;
wherein said plastic enclosure comprises a USB port such that said omnisign medical device system is able to communicate with said vital sign display device via a USB cable;
wherein said temperature sensor, said first-acoustic sensor, and said second-acoustic sensor are located about an exterior of said plastic enclosure and connected to said vital sign detection assembly;
wherein said plastic enclosure is securely mounted to said arm cuff air bladder;
wherein said arm cuff air bladder is securable about a forearm of a user via said forearm-attacher such that said temperature sensor, said first-acoustic sensor, and said second-acoustic sensor are in touch-contact with at least one vital sign detection area of said user;
wherein said arm cuff air bladder comprises a pneumatic pump comprising a 30 Vdc air pump, and at least one valve working in functional combination to measure a blood pressure of said user;
wherein said first-acoustic sensor is structured and arranged to detect a heart rate of said user by detecting a heartbeat from within a chest cavity of said chest of said user;
wherein said second-acoustic sensor is structured and arranged to detect a respiratory rate of said user by detecting a breathing pattern produced by lungs of said user from within said chest cavity;
wherein said temperature sensor is placeable against an underarm of said user for detecting a body temperature of said user;
wherein said display-device-housing, said display-device-assembly, said LCD display screen, and said display-device-assembly power supply comprises in functional combination said vital sign display device;
wherein said display-device-assembly is securely mounted inside a hollow confine of said display-device-housing;
wherein said display-device-assembly is powered by said display-device-assembly power supply;
wherein said display-device-assembly comprises a wireless transceiver module for wirelessly receiving at least one byte of data from said wireless transmitter module of said vital sign detection assembly;
wherein said LCD display screen is installed to a front of said display-device-housing; and
wherein said omnisign medical device system is structured and arranged to communicate said blood pressure, said heart rate, said respiratory rate, and said body temperature of said user to said vital sign display device for displaying said at least one vital sign of said user in real-time on said LCD display screen

19. The omnisign medical device system of claim 18 further comprising a kit including:
said vital sign detection assembly;
said vital sign display device; and
a set of user instructions.

20. A method of using an omnisign medical device system comprising the steps of:
placing a vital sign detection assembly around a forearm of a user;
adjusting a size of an arm cuff air bladder to fit said forearm of said user;
positioning a temperature sensor, a first-acoustic sensor, and a second-acoustic in touch-contact with at least one vital sign area of said user; and
monitoring at least one vital sign of said user via a vital sign display device.
